(19) United States
(12) Patent Application Publication
(10) Pub. No.: US 2013/0282033 A1
(43) Pub. Date: Oct. 24, 2013

(54) Apparatus and Method to Facilitate Position of Prosthetic Mesh

(71) Applicant: Gerardo A. Caballero, Brookfield, WI (US)

(72) Inventor: Gerardo A. Caballero, Brookfield, WI (US)

(21) Appl. No.: 13/655,976

(22) Filed: Oct. 19, 2012

Related U.S. Application Data
(60) Provisional application No. 61/549,813, filed on Oct. 21, 2011.

(51) Int. Cl. A61F 2/00

(52) U.S. Cl. CPC A61F 2/0063
USPC 606/151

(57) ABSTRACT

There is disclosed an apparatus and a method for using a hernia surgical kit in a surgical procedure. The kit includes a prosthetic mesh sheet having a smooth side and a sticky side. The kit further includes a first support rod and a second support rod.

### CROSS-REFERENCE TO RELATED PATENT APPLICATIONS

This patent application claims priority to U.S. Provisional Application No. 61/549,813, filed Oct. 21, 2011, incorporated herein in its entirety, by this reference.

### BACKGROUND OF THE INVENTION

### Field of the Invention

This invention relates to surgical appliances and more particularly to an apparatus and method to facilitate positioning of a prosthetic mesh in an animal body cavity.

It is known in the treatment of hernias that a prosthetic mesh is used to repair and stabilize a hernia in an animal, for example, a human being. The prosthetic mesh typically is composed of polyester and polylactic acid, for example a prosthetic mesh manufactured by Covidien, NA with a trade name of Paritetex®.

A typical prosthetic mesh provides one side with a smooth surface and an opposite side of the mesh including an absorbable Velcro-like side that facilitate fixation of the mesh in a given position. Such construction obviates the need for additional fixation implants with the resulting decrease in pain to the patient.

In a typical laparoscopic inguinal hernia application, because of the limited space and self-sticking properties of the prosthetic mesh, the surgeon experiences difficulty in properly placing the mesh in the body cavity of the patient. It is known to deploy a prosthetic mesh, during laparoscopic surgery, through a trocar.

It is also known that a hernia repair patch can be inserted into a body cavity deployed through a cannula.

The apparatus of the present disclosure must also be of construction which is both durable and long lasting, and it should also require little or no maintenance to be provided by the user throughout its operating lifetime. In order to enhance the market appeal of the apparatus of the present disclosure, it should also be of inexpensive construction to thereby afford it the broadest possible market. Finally, it is also an objective that all of the aforesaid advantages and objectives be achieved without incurring any substantial relative disadvantage.

### SUMMARY OF THE INVENTION

The disadvantages and limitations of the background art discussed above are overcome by the present disclosure.

There is provided a method for using a hernia surgical kit in a surgical procedure. The kit includes a prosthetic mesh sheet having a smooth side and a sticky side. The kit further includes a first support rod and a second support rod.

The method of using the surgical kit includes placing the first support rod proximate a distal edge of the sticky side of the prosthetic mesh sheet. The user then folds the distal edge over the first support rod and couples the distal edge to the sticky side forming a first fold-over retaining the first support rod within the first fold-over.

The method further includes placing the second support rod proximate a proximal edge of the prosthetic mesh sheet and folding the proximal edge over the second support rod and coupling the proximal edge to the sticky side forming a second fold-over, with the second support rod enclosed within the second fold-over.

The operator then moves the second fold-over toward the first fold-over forming a third fold-over. The second fold-over is coupled to the sticky side of the prosthetic mesh sheet immediately below the first fold-over. The third fold-over is further moved towards the first and second fold-over forming a fourth fold-over. The third fold-over is also coupled to the sticky side below the second fold-over.

Additional fold-overs can be formed by continually folding the lowest fold up towards the previous fold-overs thereby creating a compact device for insertion into a preperitoneal space in a body cavity.

Prior to insertion into the body cavity, the folded prosthetic sheet is rotated 180 degrees. The folded prosthetic sheet is then inserted into a body cavity through an umbilical port defined in the body. Once in the body cavity, the folded prosthetic sheet is unfolded by moving the fourth fold-over inside the body cavity. The third fold-over is also unfolded inside the body cavity and securing the first fold-over to the underside of the body cavity wall. Using the second fold-over with the second support rod securing the prosthetic mesh at a selected location to the underside of the body cavity wall. Once the prosthetic mesh sheet is secured inside the body cavity wall, the first and second support rods are removed. The sticky side of the prosthetic mesh sheet is secured to the body cavity wall by absorbable hooks configured to engage the body tissue. After removal of the first and second support rods, the operator, surgeon, completes the surgical procedure.

There is also provided a hernia surgical kit including a prosthetic mesh sheet. The prosthetic mesh sheet includes a first side and a second side with the sheet defined by a distal edge and a proximal edge. The surgical kit further includes a first support rod configured as an elongated body having a circular cross section and a second support rod configured as an elongated body having a circular cross section.

Each of the support rods are further configured to releasably engage the prosthetic mesh sheet and the prosthetic mesh sheet is also configured for multiple folds. In one embodiment, the first side of the prosthetic mesh sheet is configured as a sticky side composed of absorbably hooks configured to engage body tissue and the second side of the prosthetic mesh sheet is configured as a smooth side. In one embodiment, the first and second support rods are composed of a plastic polymer suitable for surgical procedures.

The apparatus of the present invention is of a construction which is both durable and long lasting, and which will require little or no maintenance to be provided by the user throughout its operating lifetime. Finally, all of the aforesaid advantages and objectives are achieved without incurring any substantial relative disadvantage.

### DESCRIPTION OF THE DRAWINGS

These and other advantages of the present disclosure are best understood with reference to the drawings, in which:

FIG. 1A-I illustrate an exemplary embodiment of a prosthetic kit including a prosthetic mesh, and a first and second support rods, and illustrating a configuration of the prosthetic kit for insertion into a pre-peritonatal space in an animal body cavity and deploying the prosthetic mesh in a position selected by an operator.

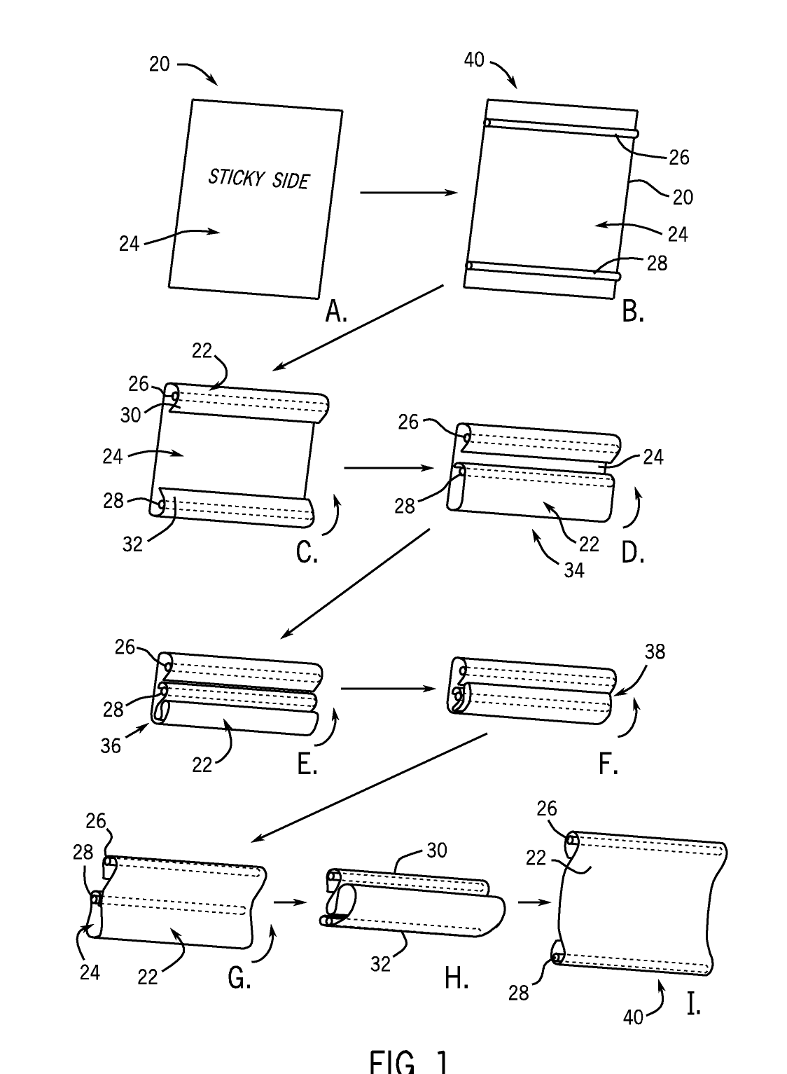

### DETAILED DESCRIPTION OF THE EXEMPLARY EMBODIMENTS

There is provided a method for using a hernia surgical kit 40 in a surgical procedure. The kit includes a prosthetic mesh sheet 20 having a smooth side 22 and a sticky side 24. The kit 40 further includes a first support rod 26 and a second support rod 28.

An example of a prosthetic mesh sheet 20 is a product, manufactured by Covidien, NA, which is a composite open skirt mesh known by the trade name of Parietex™. Such mesh is a three-dimensional mesh with a restorable collagen film. The material that composes the mesh is typically a macro porous polyester material suitable for surgical applications.

The method of using the surgical kit 40 includes placing the first support rod 26 proximate a distal edge 42 of the sticky side 24 of the prosthetic mesh sheet 20. The user then folds the distal edge 42 over the first support rod 26 and couples the distal edge 42 to the sticky side 24 forming a first fold-over 30 retaining the first support rod 26 within the first fold-over 30.

The method further includes placing the second support rod 28 proximate a proximal edge 44 of the prosthetic mesh sheet 20 and folding the proximal edge 44 over the second support rod 28 and coupling the proximal edge 44 to the sticky side 24 forming a second fold-over 32, with the second support rod 28 enclosed within the second fold-over 32.

The operator then moves the second fold-over 32 toward the first fold-over 30 forming a third fold-over 34. The second fold-over 32 is coupled to the sticky side 24 of the prosthetic mesh sheet 20 immediately below the first fold-over 30. The third fold-over 34 is further moved towards the first and second fold-over 30, 32 forming a fourth fold-over 36. The third fold-over 34 is also coupled to the sticky side 24 below the second fold-over 32. Additional fold-overs N^{th} can be formed by continually folding the lowest fold up towards the previous fold-overs thereby creating a compact device for insertion into a preperitoneal space in a body cavity.

The body cavity is defined by tissue of an animal body. Typically the animal body is a human body. The typical body cavity is the abdominal portion of the human body.

Prior to insertion into the body cavity, the folded prosthetic sheet 20 is rotated 180 degrees. The folded prosthetic sheet 20 is then inserted into a body cavity through an umbilical port defined in the body. Once in the body cavity, the folded prosthetic sheet 20 is unfolded by moving the fourth fold-over 36 (or N^{th} fold-over) inside the body cavity. The third fold-over 34 is also unfolded inside the body cavity and securing the first fold-over 30 to the underside of the body cavity wall. Using the second fold-over 32 with the second support rod 28 securing the prosthetic mesh 20 at a selected location to the underside of the body cavity wall. Once the prosthetic mesh sheet 20 is secured inside the body cavity wall, the first and second support rods 26, 28 are removed. The sticky side 24 of the prosthetic mesh sheet 20 is secured to the body cavity wall by absorbable hooks, for example self-sticking Velcro-like hooks, configured to engage the body tissue, for example transversalis fascia in the preperitoneal space in the body cavity. After removal of the first and second support rods 26, 28, the operator, surgeon, completes the surgical procedure.

There is also provided a hernia surgical kit 40 including a prosthetic mesh sheet 20. The prosthetic mesh sheet 20 includes a first side and a second side with the sheet defined by a distal edge 42 and a proximal edge 44. The surgical kit 40 further includes a first support rod 26 configured as an elongated body having a circular cross section and a second support rod 28 configured as an elongated body having a circular cross section.

Each of the support rods 26, 28 are further configured to releasably engage the prosthetic mesh sheet 20 and the prosthetic mesh sheet 20 is also configured for multiple folds. In one embodiment, the first side of the prosthetic mesh sheet 20 is configured as a sticky side 24 composed of absorbably hooks configured to engage body tissue and the second side of the prosthetic mesh sheet 20 is configured as a smooth side 22. In one embodiment, the first and second support rods 26, 28 are composed of a plastic polymer suitable for surgical procedures.

The first and second support rods 26, 28 are preferably composed of polyethylene which provides a stable but flexible cylinder to allow a physician to maneuver the two support rods within the body cavity for placement of the prosthetic mesh sheet 20. It is contemplated that other types of polymers suitable for surgical applications can compose the first and second support rods 26, 28. In an embodiment, the support rods 26, 28 define perforations on the ends of each rod to facilitate coupling and uncoupling with the mesh sheet 20.

For purposes of this disclosure, the term “coupled” means the joining of two components (electrical or mechanical) directly or indirectly to one another. Such joining may be stationary in nature or moveable in nature. Such joining may be achieved with the two components (electrical or mechanical) and any additional intermediate members being integrally formed as a single unitary body with one another or the two components and any additional member being attached to one another. Such adjoining may be permanent in nature or alternatively be removable or releasable in nature.

Although the foregoing description of the present mechanism has been shown and described with reference to particular embodiments and applications thereof, it has been presented for purposes of illustration and description and is not intended to be exhaustive or to limit the disclosure to the particular embodiments and applications disclosed. It will be apparent to those having ordinary skill in the art that a number of changes, modifications, variations, or alterations to the mechanism as described herein may be made, none of which depart from the spirit or scope of the present disclosure. The particular embodiments and applications were chosen and described to provide the best illustration of the principles of the mechanism and its practical application to thereby enable one of ordinary skill in the art to utilize the disclosure in various embodiments and with various modifications as are suited to the particular use contemplated. All such changes, modifications, variations, and alterations should therefore be seen as being within the scope of the present disclosure as determined by the appended claims when interpreted in accordance with the breadth to which they are fairly, legally, and equitably entitled.

What is claimed is:

1. A method for using a hernia surgical kit in a surgical procedure, the kit including a prosthetic mesh sheet having a smooth side and a sticky side, a first support rod and a second support rod, the method comprising:
placing the first support rod proximate a distal edge of the sticky side of the prosthetic mesh sheet;
folding the distal edge over the first support rod and coupling the distal edge to the sticky side forming a first fold-over;
placing the second support rod proximate a proximal edge of the prosthetic mesh sheet;
folding the proximal edge over the second support rod and coupling the proximal edge to the sticky side forming a second fold-over;
moving the second fold-over toward the first fold-over forming a third fold-over, wherein the second fold-over is coupled to the sticky side below the first fold-over;
moving the third fold-over toward the first and second fold-over forming a fourth fold-over, wherein the third fold-over is coupled to the sticky side below the second fold-over;
rotating the folded prosthetic sheet 180°;
inserting the folded prosthetic sheet into a body cavity through an umbilical port defined in the body;
unfolding the fourth fold-over inside the body cavity;
unfolding the third fold-over inside the body cavity and securing the first fold-over to the underside of the body cavity wall;
using the second fold-over with the second support rod securing the prosthetic mesh at a selected location to the underside of the body cavity wall;
removing the first and second support rod; and
completing the surgical procedure.

2. The method of claim 1, further comprising at least one additional fold-over forming a nth fold-over.

3. The method of claim 1, wherein the prosthetic mesh sheet is a rectangular shape.

4. The method of claim 1, wherein the sticky side of the prosthetic mesh sheet is composed of absorbable hooks configured to engage body tissue.

5. The method of claim 1, wherein the first and second support rod is composed of a plastic polymer suitable for surgical procedures.

6. The method of claim 5, wherein the plastic polymer is polypropylene.

7. The method of claim 1, wherein the body cavity is defined in a human body.

8. A hernia surgical kit comprising:
a prosthetic mesh sheet, the sheet including a first side and a second side, with the sheet defined by a distal edge and a proximal edge;
a first support rod configured as an elongated body having a circular cross-section; and
a second support rod configured as an elongated body having a circular cross-section,
wherein each of the support rods are further configured to releasably engage the prosthetic mesh sheet and the prosthetic mesh sheet is configured for multiple folds.

9. The hernia surgical kit of claim 8, wherein the first side is configured as a sticky side composed of absorbable hooks configured to engage animal body tissue and the second side is configured as a smooth side.

10. The hernia surgical kit of claim 9, wherein the animal body tissue is human body tissue.

11. The hernia surgical kit of claim 8, wherein the first and second support rod is composed of a plastic polymer suitable for surgical procedures.

12. The hernia surgical kit of claim 11, wherein the plastic polymer is polypropylene.

13. The hernia surgical kit of claim 8, wherein the prosthetic mesh sheet is a rectangular shape.
